(19) United States
(12) Patent Application Publication
(10) Pub. No.: US 2005/0107438 A1
(43) Pub. Date: May 19, 2005

(54) Pharmaceutical composition comprising 3-[(2-{[4-(Hexyloxycarbonylaminoiminomethyl) phenylamino]-methyl}-1-methyl-1H-benzimidazol-5-carbonyl)-pyridin-2-yl-amino]-propionic acid ethyl ester or a salt therefore

(75) Inventors: Guido Bernhard Edmund Radtke, Biberach (DE); Ulrich Busch, Biberach (DE); Achim Sauer, Ravensburg-Torkenweiler (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 10/925,598

(22) Filed: Aug. 25, 2004

Related U.S. Application Data
(60) Provisional application No. 60/505,495, filed on Sep. 24, 2003.

(30) Foreign Application Priority Data
Sep. 3, 2003 (DE) 103 41 043

(51) Int. Cl. A61K 31/4439

(52) U.S. Cl. 514/338

(57) ABSTRACT

A pharmaceutical composition comprising: ethyl 3-[(2-{[4-(hexyloxycarbonylaminoiminomethyl)phenylamino]methyl}-1-methyl-1H-benzimidazole-5-carbonyl)pyridin-2-ylamino]propionate or a pharmaceutically acceptable salt thereof; and a pharmaceutically acceptable lipophilic liquid, solid, or semi-solid carrier system.

### RELATED APPLICATIONS

This application claims benefit of U.S. Ser. No. 60/505,495, filed Sep. 24, 2003, and claims priority to German Application No. 103 41 043.0, filed Sep. 3, 2003, each of which is hereby incorporated by reference in its entirety.

### FIELD OF THE INVENTION

The invention relates to a formulation for oral administration of the active substance ethyl 3-[(2-{[4-(hexyloxycarbonylaminoiminomethyl)phenylamino]methyl}-1-methyl-1H-benzimidazole-5-carbonyl)pyridin-2-ylamino]propionate and the pharmacologically acceptable salts thereof. This active substance with the chemical formula 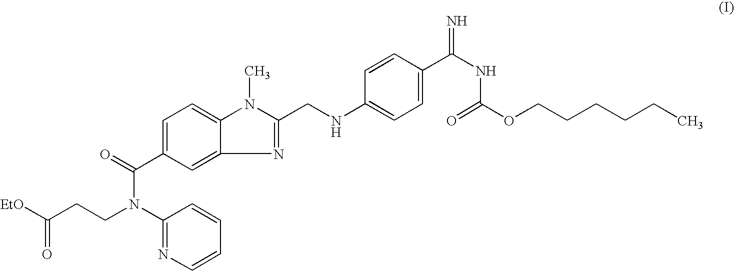
is already known from WO 98/37075 (corresponding to U.S. Pat. Nos. 6,087,380; 6,469,039; 6,414,008; and 6,710,055, which are each hereby incorporated by reference), in which compounds with a thrombin-inhibiting effect and a thrombin time-prolonging activity are disclosed, under the name 1-methyl-2-[N-[4-(N-n-hexyloxycarbonyl-amidino)phenyl]aminomethyl]benzimidazol-5-yl-carboxylic acid-N-(2-pyridyl)-N-(2-ethoxycarbonylethyl)amide. The compound of formula (I) is a double prodrug of the compound of formula (II) 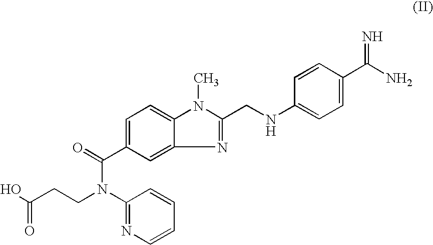
i.e., the compound of formula (I) is only converted into the actual effective compound, namely the compound of formula (II), in the body. The main field of application of the compound of chemical formula (I) is the post-operative prophylaxis of deep vein thrombosis.

The aim of the invention is to provide an improved formulation for the oral administration of the compound of formula (I) and the pharmacologically acceptable salts thereof (also referred to hereinafter as the “active substance”).

Surprisingly it has now been found that an anhydrous formulation which contains the active substance ethyl 3-[(2-{[4-(hexyloxycarbonylaminoiminomethyl)phenylamino]-methyl}-1-methyl-1H-benzimidazole-5-carbonyl)pyridin-2-ylamino]propionate or one of the pharmaceutically acceptable salts thereof in dispersed form in a lipophilic, pharmaceutically acceptable carrier system, leads to oral preparations which have significantly better properties. The carrier system may be both solid, semi-solid, and liquid.

The formulation according to the invention comprises: 5 wt. % to 40 wt. % of the active substance, 55 wt. % to 95 wt. % of a pharmaceutically acceptable, lipophilic carrier system, and optionally 0 wt. % to 5 wt. % of one or more stabilizers, the amounts of ingredients adding up to a total of 100 wt. %.

A formulation containing stabilizer is preferred.

A preferred formulation according to the invention comprises: 8 wt. % to 40 wt. % of the active substance, 60 wt. % to 92 wt. % of a pharmaceutically acceptable, lipophilic carrier system, and optionally 1 wt. % to 4 wt. % of one or more stabilizers, the amounts of ingredients adding up to a total of 100 wt. %.

A particularly preferred formulation according to the invention comprises: 16 wt. % to 36 wt. % of the active substance, 61 wt. % to 81 wt. % of a pharmaceutically acceptable, lipophilic carrier system, and optionally 2 wt. % to 3 wt. % of one or more stabilizers, the amounts of ingredients adding up to a total of 100 wt. %.

The finished formulation may be packed into hard or soft capsules made, for example, from gelatine, hydroxypropylmethylcellulose (HPMC), polulan, starch, modified starch, algae, or other material which is broken down in the gastrointestinal tract.

By the active substance is meant ethyl 3-[(2-{[4-(hexyloxycarbonylaminoiminomethyl)-phenylamino]methyl}-1-methyl-1H-benzimidazole-5-carbonyl)pyridin-2-ylamino]propionate or one of the pharmaceutically acceptable salts thereof; pharmaceutically acceptable salts of ethyl 3-[(2-{[4-(hexyloxycarbonylaminoiminomethyl)phenylamino]methyl}-1-methyl-1H-benzimidazole-5-carbonyl)pyridin-2-ylamino]propionate are preferably used. Ethyl 3-[(2-{[4-(hexyloxycarbonylaminoiminomethyl)phenylamino]methyl}-1-methyl-1H-benzimidazole-5-carbonyl)pyridin-2-ylamino]propionate methanesulfonate is particularly preferred. The active substance may be ground up before use. Most particularly preferred is ethyl 3-[(2-{[4-(hexyloxycarbonylaminoiminomethyl)phenylamino]methyl}-1-methyl-1H-benzimidazole-5-carbonyl)pyridin-2-ylamino]propionate methanesulfonate in ground form.

The pharmaceutically acceptable, lipophilic carrier systems for the purposes of the invention include:

(a) microemulsion concentrates such as, for example, macrogol glycerol laurates according to Ph. Eur. NT 2001; and

(b) low-melting lipophilic systems comprising:
(i) 20 wt. % to 90 wt. % (based on the finished formulation) of a lipophilic component, and
(ii) 0 wt. % to 90 wt. % (based on the finished formulation) of a surfactant compound.

Preferably microemulsion concentrates are used.

A preferred carrier system according to (b) comprises:
(i) 40 wt. % to 70 wt. % (based on the finished formulation) of a lipophilic component, and
(ii) 5 wt. % to 30 wt. % (based on the finished formulation) of a surfactant compound.

A preferred embodiment of the low-melting lipophilic systems according to (b) relates to those systems which have been homogenized under high pressure.

One advantage of the formulation according to the invention is that the active substance is chemically and physically stabilized thereby. Additionally, the formulation is characterized by good bioavailability of the active substance.

Preferred microemulsion concentrates are polyglycolized glycerides sold under the GELUCIRE® trademark by Gattefossé, while GELUCIRE® 44/14 (lauroyl macrogol-32 glycerides) is particularly preferred, i.e., the GELUCIRE® product with a melting point of 44° C. and an HLB value (hydrophilic/lipophilic balance value) of 14.

Products sold under the GELUCIRE® trademark are polyglycolized glycerides which are prepared by alcoholysis of natural oils with polyoxyethylene glycols. They are mixtures of monoesters, diesters, and/or triesters of glycerides of long-chained fatty acids with 8 to 18 carbon atoms and polyethyleneglycol mono- and/or diesters of long-chained fatty acids. These preparations have a wide range of melting points between about 33° C. and 64° C. and a wide range of HLB values of between about 1 and 14. For the present invention, GELUCIRE® products with an HLB value of 10-14 are of particular interest.

By a low-melting system is meant a system with a melting point below 50° C.

The lipophilic component (i) may consist of fats and oils such as, for example, hard fat (e.g., WITEPSOL® W 45), neutral oil, olive oil, sunflower seed oil, groundnut oil, wheat germ oil, and castor oil. Neutral oil is preferred.

The surfactant compound (ii) used may be, for example, lipophilic or hydrophilic emulsifiers such as perhaps mono- and di-glycerides of fatty acids with 8 to 18 carbon atoms and polysorbates (e.g., polysorbate 80 [polyoxyethylene(20)sorbitan monooleate), ethoxylates of fatty alcohols such as that sold under the CREMOPHOR® trademark, or hydrogenated castor oil, e.g., RH 40 (macrogol glycerol hydroxystearate)), or tagates (polyoxyethylene glycerol monofatty acid esters, e.g., tagate S2=polyoxyethylene(20) glycerol stearate).

The stabilizers used may be antioxidant adjuvants, such as, e.g., tocopherols, butylhydroxyanisole (BHA), butylhydroxytoluene (BHT), ascorbic acid or gallic acid esters, nordihydroguaiaretic acid (NDGA), or other compounds such as, e.g., flavonoids. Tocopherols, butylhydroxyanisol (BHA), and butylhydroxytoluene (BHT) are preferred according to the invention. The use of stabilizers serves to prolong the shelf life of the finished product. Tocopherol is normally used in a ratio by weight of 1:412 (tocopherol:active substance), while BHT is normally used in a ratio by weight of 1:2306 (BHT:active substance).

It is also advantageous to use pH stabilizers such as, e.g., weak organic or inorganic bases (e.g., sodium hydrogen carbonate) as well as water-binding excipients which also act as stabilizers (e.g., dried β-cyclodextrin; β-cyclodextrin with a water content of 4% is preferred).

By a suitable choice of composition, particularly by the choice of surfactant compound and by varying the particle size of the active substance, it is readily possible for the skilled person to control the release profile of the formulation according to the invention over a wide range. Thus, depending on the desired product qualities, both instant release formulations and pharmaceutical compositions with modified release may be prepared.

The active substance dispersion may be prepared by the process described hereinafter:

The liquid (molten) carrier system is placed at 50° C. to 70° C. in a reaction vessel and optionally stabilizers such as, e.g., antioxidants and/or pH stabilizers as well as internal drying agents are dissolved or suspended therein. Then the active substance is incorporated with stirring and the dispersion is homogenized. The dispersion is then degassed and can then be packed into hard or soft capsules, for example. For this purpose, a quantity of dispersion corresponding to the dosage is packed into hard capsules, for example, using a standard capsule filling machine. Examples of suitable hard capsules include hard gelatine capsules or hard capsules made of hydroxypropylmethylcellulose (HPMC). The content of active substance in the pharmaceutical composition is 5 wt. % to 40 wt. %, preferably 8 wt. % to 36 wt. % in the solidified melt, based on the methanesulfonate of the compound of formula (I).

Unless otherwise stated, the percentages given are percent by weight in each case. Unless otherwise stated, all the amounts of active substance content specified are based on the methanesulfonate of formula (I).

The dose when administered orally is conveniently 25 mg to 300 mg of the active substance base (per capsule), preferably 50 mg to 200 mg, once or twice a day in each case.

Clinical Trials

In the first clinical trials on test subjects with conventional tablets containing the compound of formula (I) it was found that highly variable plasma levels occurred, ranging to individual cases of malabsorption.

Composition of the conventional tablet:

| | Ingredient | mg/tablet |
|---|---|---|
| Core | Mesylate of the compound | 57.7 |
| | of formula (I) | |
| | Lactose monohydrate | 58.0 |
| | Microcrystalline cellulose | 48.3 |
| | Crospovidone | 3.4 |
| | magnesium stearate | 2.6 |
| Film coating | Polyethyleneglycol 6000 | 0.56 |
| | Titanium dioxide | 0.80 |
| | Talc | 0.64 |
| | hydroxypropylmethylcellulose | 1.92 |
| | iron oxide yellow | 0.08 |
| | Total | 174.0 |

The variability of the plasma levels is significantly less after the administration of the compound of formula (I) as an orally administered acidic solution; no malabsorption was observed. However, the acidic solution has an unpleasant taste and is unstable, which makes it unsuitable for commercial purposes.

Composition of the acidic solution:

| Ingredient | per dose/bottle |
|---|---|
| mesylate of the compound | 57.70 mg |
| of formula (I) | |
| mannitol | 942.30 mg |
| tartaric acid | 110.00 mg |
| purified water | 99.80 g |

Investigations have shown that the compound of formula (I) dissolves relatively easily in water at low pH values, whereas at pH levels above 5 it is virtually insoluble according to the definition of the European Pharmacopoeia. Therefore, pantoprazole which serves to raise the pH of the stomach was given to the test subjects in one branch of treatment in the clinical trials.

For example, the pharmaceutical composition according to Example 1 was tested for its bioavailability compared with the acidic solution.

To do this, the formulation prepared according to Example 1 containing 50 mg of active substance base per capsule (corresponding to 57.66 mg of the mesylate of the compound of formula (I)) was clinically tested on a total of 12 test subjects for its bioavailability. In one branch of treatment, the test subjects were given the composition orally on an empty stomach without any pre-treatment. In another branch of treatment, the same test subjects were pre-treated with 40 mg of pantoprazole b.i.d. (twice a day) by oral route to increase the gastric pH before oral administration of the composition; the treatment with pantoprazole was continued during the administration of the formulation according to the invention. The treatment lasted 3 days in each case.

The extent of absorption was determined by quantitative measurement of the excretion of the active metabolite of formula (II) in the urine.

The relative bioavailability (based on the area under the plasma concentration-time curve) was 116% with pre-treatment with pantoprazole and 131% without pre-treatment with pantoprazole, compared with the acidic solution (100% in each case). Thus, the bioavailability of the formulation according to the invention was further improved compared with the acidic solution.

The clinical trials demonstrate another advantage of the formulation according to the invention containing the compound of formula (I), which consists in ensuring a sufficient bioavailability of the active substance, which is better than that of a conventional pharmaceutical preparation and is also largely independent of the gastric pH, reducing fluctuations in the bioavailability of the active substance and preventing malabsorption. Another advantageous property of the pharmaceutical composition according to the invention is its suitability for all patients, i.e., including those people in whom the pH of the stomach is elevated as a result of normal physiological variability, by illness or by co-medication with drugs which raise the pH of the stomach.

The Examples that follow are intended to illustrate the invention:

EXAMPLE 1

| | amount/ | amount/ |
|---|---|---|
| Ingredient | capsule (mg) | capsule (mg) |
| active substance (mesylate of the | 57.66 | 115.32 |
| compound of formula (I)) | | |
| macrogol glycerol laurate Ph. Eur. | 292.34 | 584.68 |
| Total | 350.00 | 700.00 |

EXAMPLE 2

| | amount/ | amount/ |
|---|---|---|
| Ingredient | capsule (mg) | capsule (mg) |
| active substance (mesylate of the | 57.66 | 230.64 |
| compound of formula (I)) | | |
| macrogol glycerol laurate Ph. Eur. | 117.2. | 468.8 |
| α-tocopherol | 0.14 | 0.56 |
| Total | 175.00 | 700.00 |

EXAMPLE 3

| Ingredient | amount/capsule (mg) |
|---|---|
| active substance (mesylate of the | 115.32 |
| compound of formula (I)) | |
| butylhydroxytoluene (BHT) | 0.05 |
| macrogol glycerol laurate Ph. Eur. | 234.63 |
| Total | 350.00 |

EXAMPLE 4

| Ingredient | amount/capsule (mg) |
|---|---|
| active substance (mesylate of the | 172.98 |
| compound of formula (I)) | |
| macrogol glycerol laurate Ph. Eur. | 301.91 |
| α-tocopherol | 0.42 |
| predried β-cyclodextrin with 4% water | 12.19 |
| Total | 487.50 |

EXAMPLE 5

| | amount/ | amount/ |
|---|---|---|
| Ingredient | capsule (mg) | capsule (mg) |
| active substance (mesylate of the | 172.98 | 230.64 |
| compound of formula (I)) | | |
| macrogol glycerol laurate Ph. Eur. | 314.10 | 418.80 |
| α-tocopherol | 0.42 | 0.56 |
| Total | 487.50 | 650.00 |

EXAMPLE 6

| Ingredient | amount/capsule (mg) |
|---|---|
| active substance (mesylate of the | 57.66 |
| compound of formula (I)) | |
| neutral oil | 105.60 |
| Tween 80 (polysorbate 80) | 11.74 |
| Total | 175.00 |

### EXAMPLE 7

### Preparation of ethyl 3-[(2-{[4-(hexyloxycarbonylaminoiminomethylphenylamino]methyl}-1-methyl-1H-benzimidazole-5-carbonyl)pyridin-2-ylamino]propionate methanesulfonate

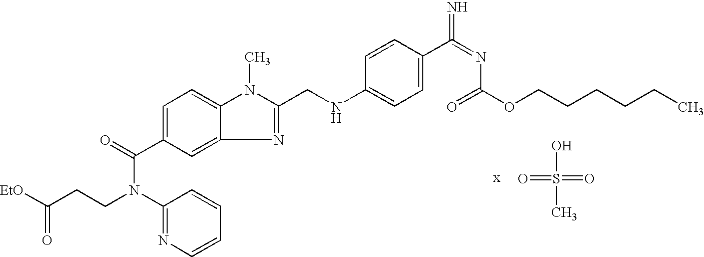

A solution of 5.0 mmol of methanesulfonic acid in 25 mL of ethyl acetate was added dropwise with stirring, at ambient temperature, to a solution of 3139 mg (5.0 mmol) of ethyl 3-[(2-{[4-(hexyloxycarbonylaminoiminomethyl)phenylamino]methyl}-1-methyl-1H-benzimidazole-5-carbonyl)pyridin-2-ylamino]propionate base (prepared as described in WO 98/37075), in 250 mL of ethyl acetate. After a few minutes, the product began to crystallize out. It was stirred for a further hour at ambient temperature and then for a further hour while cooling with ice, then the precipitate was suction filtered, washed with approximately 50 mL of ethyl acetate and 50 mL diethyl ether, and dried at 50° C. in the circulating air drying cupboard. Yield: 94% of theory; melting point: 178° C.-179° C.; C₃₄H₄₁N₇O₅×CH₄SO₃ (723.86); elemental analysis: calc.: C 58.07%, H 6.27%, N 13.55%, S 4.43%; found: C 58.11%, H 6.30%, N 13.50%, S 4.48%.

1. A pharmaceutical composition comprising:
(a) ethyl 3-[(2-{[4-(hexyloxycarbonylaminoiminomethyl)phenylamino]methyl}-1-methyl-1H-benzimidazole-5-carbonyl)pyridin-2-ylamino]propionate or a pharmaceutically acceptable salt thereof; and
(b) a pharmaceutically acceptable lipophilic liquid, solid, or semi-solid carrier system.

2. A pharmaceutical composition comprising:
(a) 5 wt. % to 40 wt. % of ethyl 3-[(2-{[4-(hexyloxycarbonylaminoiminomethyl)-phenylamino]methyl}-1-methyl-1H-benzimidazole-5-carbonyl)pyridin-2-ylamino]propionate or a pharmaceutically acceptable salt thereof; and
(b) 55 wt. % to 95 wt. % of a pharmaceutically acceptable lipophilic liquid, solid, or semi-solid carrier system; and
(c) 0 wt. % to 5 wt. % of one or more stabilizers,
wherein the amounts of ingredients total 100 wt. %.

3. The pharmaceutical composition according to claim 1, wherein the pharmaceutically acceptable lipophilic carrier system is a microemulsion concentrate.

4. The pharmaceutical composition according to claim 2, wherein the pharmaceutically acceptable lipophilic carrier system is a microemulsion concentrate.

5. The pharmaceutical composition according to claim 3, wherein the microemulsion concentrate is a macrogol glycerol laurate according to Ph. Eur. NT 2001.

6. The pharmaceutical composition according to claim 4, wherein the microemulsion concentrate is a macrogol glycerol laurate according to Ph. Eur. NT 2001.

7. The pharmaceutical composition according to claim 5, wherein the macrogol glycerol laurate is lauroyl macrogol-32 glycerides.

8. The pharmaceutical composition according to claim 6, wherein the macrogol glycerol laurate is lauroyl macrogol-32 glycerides.

9. The pharmaceutical composition according to claim 1, wherein the lipophilic carrier system is a low-melting lipophilic system comprising a lipophilic component which optionally contains one or more surfactant compounds.

10. The pharmaceutical composition according to claim 2, wherein the lipophilic carrier system is a low-melting lipophilic system comprising a lipophilic component which optionally contains one or more surfactant compounds.

11. The pharmaceutical composition according to one of claims 1 to 10, wherein the amount of ethyl 3-[(2-{[4-(hexyloxycarbonylaminoiminomethyl)phenylamino]methyl}-1-methyl-1H-benzimidazole-5-carbonyl)pyridin-2-ylamino]propionate or a salt thereof in the pharmaceutical composition is 8 wt. % to 40 wt. % based on ethyl 3-[(2-{[4-(hexyloxycarbonylaminoiminomethyl)phenylamino]methyl}-1-methyl-1H-benzimidazole-5-carbonyl)pyridin-2-ylamino]propionate methanesulfonate.

12. A hard capsule containing the pharmaceutical composition according to one of claims 1 to 10.

13. The hard capsule according to claim 12, wherein the hard capsule is a hard gelatine capsule or a hard capsule made from hydroxypropylmethylcellulose.

14. The pharmaceutical composition according to one of claims 1 to 10, wherein the ethyl 3-[(2-{[4-(hexyloxycarbonylaminoiminomethyl)-phenylamino]methyl}-1-methyl-1H-benzimidazole-5-carbonyl)pyridin-2-ylamino]propionate or a pharmaceutically acceptable salt thereof is ethyl 3-[(2-{[4-(hexyloxycarbonylaminoiminomethyl)phenylamino]methyl}-1-methyl-1H-benzimidazole-5-carbonyl)pyridin-2-ylamino]propionate mesylate.
